(19) United States
(12) Patent Application Publication
(10) Pub. No.: US 2002/0009782 A1
(43) Pub. Date: Jan. 24, 2002

(54) Heparin and heparan sulfate derived oligosaccharides and a method for their manufacture

(76) Inventor: Daphna Miron, Rehovot (IL)

Correspondence Address: SOL SHEINBEIN, c/o ANTHONY CASTORINA, SUITE 207, 2001 JEFFERSON DAVIS HIGHWAY, ARLINGTON, VA, 22202, US

(21) Appl. No.: 09/865,677

(22) Filed: Sep. 4, 2001

Related U.S. Application Data
(62) Division of application No. 09/324,508, filed on Jun. 3, 1999.

(51) Int. Cl. C12P 19/26; C08B 37/10

(52) U.S. Cl. 435/84; 536/21

(57) ABSTRACT

A method of preparing low molecular weight heparin or low molecular weight heparan sulfate, the method is effected by digesting unfractionated or partially fractionated heparin or heparan sulfate having a given preponderant molecular mass with heparanase, thereby obtaining beparin or heparan sulfate of a lower preponderant molecular mass. A product obtained by implementing the method and a pharmaceutical composition including the latter are also described.

This is a divisional of U.S. patent application Ser. No. 09/324,508, filed Jun. 3,1999.

### FIELD AND BACKGROUND OF THE INVENTION

The present invention relates to heparin and heparan sulfate oligosaccharides and to a method for their manufacture. More particularly, the present invention relates to heparin and heparan sulfate oligosaccharides produced by digestion with a heparanase, such as recombinant human heparanase.

Use of Low Molecular Weight Heparins for Treatment and Prevention of Thromboembolism:

Low molecular weight heparins (LMWHs) are fragments of unfractionated heparin produced by controlled enzymatic, chemical or physical depolymerization. These processes yield chains with a mean molecular weight of about 5,000 Daltons. LMWHs have recently established their niche as an important class of antithrombotic compounds. They have replaced unfractionated heparin in many parts of Europe, but are only now finding their place in North America. LMWHs produce a more predictable anticoagulant response than unfractionated heparin, reflecting their better bioavailability, longer half-life and dose-independent clearance. This advantage allows treatment with LMWHs with no laboratory monitoring during the treatment. Due to the fact that LMWHs cause less heparin-induced thrombocytopenia and possibly less osteoporosis, the use of LMWHs is likely to increase in the coming years.

In recent years the interest has been centered on heparin fragments with a high XaI/antithrombin activity, as they have good antithrombotic efficiency and at the same time no or little tendency to cause bleeding complications. The main difference between unfractionated heparin and LMWHs is their relative inhibitory activity against factor Xa and thrombin. Any heparin chain containing a specific pentasaccharide can inhibit the action of factor Xa, simply by binding antithrombin and causing a conformational change. In contrast, to inactivate thrombin, heparin must bind to both antithrombin and thrombin by a heparin chain composed of at least 18 saccharide units—about 5,400 Daltons.

LMWHs also cause less bleeding than unfractionated heparins, due to a lower inhibition of platelet function, because they do not increase microvascular permeability and because of their lower affinity for endothelial cells, von Willebrand factor and platelets.

LMWH was found to be more effective for the prevention of thrombosis in general surgery, orthopedic surgery, acute spinal injury, and multiple trauma, and it can be given once daily while causing fewer hematomas without monitoring required during the treatment. In treatment of venous thromboembolism and unstable angina, LMWHs are at least as safe and effective as unfractionated heparin, but can be given subcutanously without monitoring (1).

Use of LMWHs as Heparanase Inhibitors:

HSPGs (heparan sulfate proteoglycans) have a central role in embryonic morphogenesis, angiogenesis, metastasis, neurite outgrowth and tissue repair (2-6). The heparan sulfate (HS) chains, unique in their ability to bind a multitude of proteins, ensure that a wide variety of effector molecules cling to the cell surface (5-7). The ability of HSPGs to interact with extra-cellular matrix (ECM) macromolecules such as collagen, laminin and fibronectin, and with different attachment sites on plasma membranes suggests a key role for this proteoglycan in the self-assembly and insolubility of ECM components, as well as in cell adhesion and locomotion. Cleavage of HS may therefore result in disassembly of the subendothelial ECM and hence may play a decisive role in extravasation of normal and malignant blood-borne cells (8-10). HS catabolism is observed in inflammation, wound repair, diabetes, and cancer metastasis, suggesting that enzymes, which degrade HS, play important roles in pathologic processes.

ECM HSPGs also provide a natural storage depot for bFGF and possibly other heparin-binding growth promoting factors. Heparanase mediated release of active bFGF from its storage within ECM may therefore provide a novel mechanism for induction of neovascularization in normal and pathological situations (7, 11).

Heparanase activity has been shown to correlate with the ability of activated cells of the immune system to leave the circulation and elicit both inflammatory and autoimmune responses. Interaction of platelets, granulocytes, T and B lymphocytes, macrophages and mast cells with the subendothelial ECM is associated with degradation of heparan sulfate (HS) by heparanase activity (8).

LMWHs have previously been shown to inhibit heparanase activity.

Treatment of experimental animals with the LMWH laminarin sulfate markedly reduced (>90%) the incidence of lung metastases induced by B16 melanoma, Lewis lung carcinoma and mammary adenocarcinoma cells (9,10,12), indicating that LMWHs may be applied to inhibit tumor cell invasion and metastasis.

Treatment of experimental animals with a non-anticoagulant species of LMWH markedly reduced the incidence of experimental autoimmune encephalomyelitis (EAE), adjuvant arthritis and graft rejection (8, 13), indicating that LMWHs may be applied to inhibit autoimmune and inflammatory diseases (8,13).

LMWHs may also be used to inhibit pathological processes involving the induction of TNFα Secretion. TNFα is involved in pathogenesis, graft rejection, undesirable inflammatory condition in autoimmune diseases, vasculitis and atherosclerosis. It may also have roles in heart failure, in the response to cancer and in anorexia nervosa.

LMWHs may allow regulation of secretion of TNFα through inhibition of its release by T-lymphocytes (14).

Production of Low Molecular Weight Heparins:

LMWHs are currently produced in several different ways: (i) enrichment of LMWH present in standard heparin by fractionation; ethanol and or molecular sieving e.g., gel filtration or membrane filtration; (ii) controlled chemical depolymerization (by nitrous acid,β-elimination or periodate oxidation); and (iii) enzymatic depolymerization by heparinases. The conditions for depolymerization can be carefully controlled to yield products of desired molecular weights. Nitrous acid depolymerization is commonly used.

Also employed is depolymerization of the benzylic ester of heparin by β-elimination, which yields the same type of fragment as enzymatic depolymerization using heparinases.

LMWHs with low anticoagulant activity and retaining basic structure can be prepared by depolymerization using periodate oxidation.

Several LMWHs are available commercially: (i) FRAGMIN with molecular weight of 4000-6000 Daltons is produced by controlled nitrous acid depolymerization of sodium heparin from porcine intestinal mucosa by Kabi Pharmacia Sweden (see also U.S. Pat. No. 5,686,431 to Cohen et al.).

FRAXIPARIN and FRAXIPARINE with an average molecular weight of 4,500 Daltons are produced by fractionation or controlled nitrous acid depolymerzation, respectively, of calcium heparin from porcine intestinal mucosa by Sanofi (Chaoy laboratories).

LOVENOX (ENOXAPARIN and ENOXAPARINE) is produced by depolymerization of sodium heparin from porcine intestinal mucosa using β-elimination by Farmuka SF France and distributed by Rhone-Poulenc under the trade names CLEXANE and LOVENOX.

LOGIPARIN (LHN-1, Novo, Denmark) with a molecular weight of 600 to 20,000 and with more than 70% between 1500 and 10,000 Daltons is produced by enzymatic depolymerization of heparin from intestinal mucosa, using heparinase. See also U.S. Pat. No. 5,534,619 to Wakefield et al.

Mechanisms for Enzymatic Degradation of Heparin and Heparan Sulfate:

Heparin and heparan sulfate may be degraded enzymatically either by hydrolases (EC 3.2.1.-) or by lyases (EC 4.2.2.) from bacterial origin known as heparinases (15). The difference between eliminative cleavage by lyases and hydrolytic cleavage by lyases is that in the eliminative mechanism, the C5 hydrogen of uronic acid is abstracted, forming an unsaturated C4—C5 bond, whereas in the hydrolytic mechanism a proton is donated to the glycosidic bond, breaking the glycosidic oxygen and creating an O5 oxonium ion followed by water addition, which neutralizes the oxonium ion and saturates all carbons (16). The lyases can only cleave linkages on the non-reducing side of uronic acids, as the carboxylic group of uronic acid participates in the reaction. The hydrolases, on the other hand, can be specific for either of the two bonds in the repeating disaccharides (15).

To differentiate between lyase and hydrolase activity of a GAG degrading enzyme, a simple measurement of product formation based on the 232 nm absorbance the Δ4,5 unsaturated bond of GAG cleavage by lyases may be performed (17).

In a recent study, cleavage site of heparanase preparations from human platelets and from hepatoma cells was determined based on identification of fragments generated by cleavage of a well defined heparin octasaccharides. The results of this study show that heparanase from both sources is a β-ο-glucoronidase, i.e., a hydrolase (19).

### SUMMARY OF THE INVENTION

According to one aspect of the present invention there is provided a method of preparing low molecular weight heparin or low molecular weight heparan sulfate, the method comprising the step of digesting unfractionated or partially fractionated heparin or heparan sulfate having a given preponderant molecular mass with heparanase, thereby obtaining heparin or heparan sulfate of a lower preponderant molecular mass.

According to another aspect of the present invention there is provided a low molecular weight heparin or heparan sulfate preparation obtained by digesting unfractionated or partially fractionated heparin or heparan sulfate having a given preponderant molecular mass with heparanase, thereby obtaining heparin or heparan sulfate of a lower preponderant molecular mass.

According to yet another aspect of the present invention there is provided a pharmaceutical composition or medical device comprising as an active ingredient the low molecular weight heparin or heparan sulfate preparation described above and a pharmaceutically acceptable carrier.

According to further features in preferred embodiments of the invention described below, the heparanase is recombinant.

According to still further features in the described preferred embodiments the heparanase is of human origin.

According to still further features in the described preferred embodiments digesting unfractionated or partially fractionated heparin or heparan sulfate is continued until the lower preponderant molecular mass reaches between 1,000 Daltons and 10,000 Daltons.

According to still further features in the described preferred embodiments digesting unfractionated or partially fractionated heparin or heparan sulfate is continued until the lower preponderant molecular mass reaches between 3,000 Daltons and 6,000 Daltons.

According to still further features in the described preferred embodiments when sufficient digestion of the unfractionated or partially fractionated heparin or heparan sulfate has taken place, the heparanase is inactivated.

According to still further features in the described preferred embodiments the method further comprising the step of precipitating the heparin or heparan sulfate of the lower preponderant molecular mass.

According to still further features in the described preferred embodiments precipitation is effected by ethanol and salt.

According to still further features in the described preferred embodiments the method further comprising the step of size fractionating the heparin or heparan sulfate of the lower preponderant molecular mass and collecting low molecular weight heparin or heparan sulfate of a specific molecular mass range.

According to still further features in the described preferred embodiments the specific range is selected from the group consisting of 1,000-10,000 and 3,000-6,000 Daltons for at least 90% of the heparin or heparan sulfate molecules.

The present invention successfully addresses the shortcomings of the presently known configurations by providing a new method of producing low molecular weight heparin and heparan sulfate using heparanase preferably of a human source which best mimics the human in vivo process of heparin and heparan sulfate degradation.

### BRIEF DESCRIPTION OF THE DRAWINGS

The invention is herein described, by way of example only, with reference to the accompanying drawings, wherein:

FIG. 1 demonstrates the determination of lyase activity of heparanase and heparinase I. The formation of a Δ4,5 unsaturated bond of heparin cleavage by a lyase was measured as absorbance at 232 nm. Heparinase I activity served as a positive control.

FIG. 2 demonstrates product formation due to recombinant human heparanase and heparinase I activity using the dimethylmethylene blue (DMB) colorimetric assay. The incubation period for the activity in this assay was similar to the incubation time for measurement of absorbance at 235 nm, to ensure the recombinant human heparanase was active during this period of time.

FIG. 3 demonstrates a polyacrylamide gel electrophoresis analysis of heparanase derived products. The products of heparanase activity (lane 2) were separated on a 4-20% polyacrylamide gel and stained with DMB. The size of the products was compared to unfractionated heparin (lane 1) and low molecular weight heparin standards with an average size of 6,000 Daltons (lane 3) and 3,000 Daltons (lane 4).

### DESCRIPTION OF THE PREFERRED EMBODIMENTS

The present invention is of heparin and heparan sulfate oligosacharides and to a method for their manufacture which can be used as therapeutic agents and as a source for oligosaccharides exhibiting unique pharmacological properties.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Thus, according to one aspect of the present invention there is provided a method of preparing low molecular weight heparin or low molecular weight heparan sulfate. The method according to this aspect of the present invention is effected by digesting unfractionated or partially fractionated heparin or heparan sulfate having a given preponderant molecular mass with heparanase, thereby obtaining heparin or heparan sulfate of a lower preponderant molecular mass.

According to another aspect of the present invention there is provided a low molecular weight heparin or heparan sulfate preparation obtained by digesting unfractionated or partially fractionated heparin or heparan sulfate having a given preponderant molecular mass with heparanase, thereby obtaining heparin or heparan sulfate of a lower preponderant molecular mass.

According to yet another aspect of the present invention there is provided a pharmaceutical composition or medical device comprising as an active ingredient the low molecular weight heparin or heparan sulfate preparation described herein and a pharmaceutically acceptable carrier.

The term “preponderant molecular mass” is used to denote the molecular mass of the constituents of the heparin or heparan sulfate which correspond to the peak of the chromatographic profile obtained by exclusion chromatography, using a UV detector at 205 nm. To this effect see U.S. Pat. No. 5,599,801.

The heparanase used to implement the method according to the first aspect of the present invention and to obtain the low molecular weight heparin or heparan sulfate preparation according to the second aspect of the present invention can be of any source, both natural or recombinant.

The cloning of the human and mouse heparanase-encoding gene, its genetic manipulation, methods of isolating and genetically manipulating homologous genes from other species, its expression in various expression systems, its purification and its natural or artificial activation are described in detail in U.S. Pat. application Ser. Nos. 08/922,170; 09/071,618; 09/109,386; 09/258,892 and 09/260,038 and in PCT/US98/17954 and PCT/US98/09256, all of which are incorporated herein by reference. The purification of active heparanase from a natural source is described, for example, in U.S. Pat. No. 5,362,641 and in reference 18. Each of the heparanases described above can be used to implement the method according to the first aspect of the present invention and to obtain the low molecular weight heparin or heparan sulfate preparation according to the second aspect of the present invention.

According to a preferred embodiment of the present invention, when sufficient digestion of the unfractionated or partially fractionated heparin or heparan sulfate has taken place, the heparanase is inactivated. Inactivation of the heparanase can be effected in any one of a plurality of techniques employed in the art for enzyme inactivation, including, but not limited to, heat inactivation, dilution, e.g., by dialysis, exposure to extreme pH followed, for example, by neutralization, and the like. The time required for sufficient digestion of the unfractionated or partially fractionated heparin or heparan sulfate will depend on several factors, including, but not limited to, active heparanase concentration, temperature, pH and solutes other than the enzyme and substrate. One ordinarily skilled in the art would know how to modify these factors so as to obtain controlled and repetitive performance.

It will be appreciated by one ordinarily skilled in the art that the heparanase enzyme can be bound to a solid matrix and that the time of digestion of the unfractionated or partially fractionated heparin or heparan sulfate can thus by controlled by controlling the exposure time of the unfractionated or partially fractionated heparin or heparan sulfate to the solid matrix.

Monitoring the digestion reaction according to the present invention can be effected by periodic sampling and one of a plurality of known techniques, including, but not limited to, high performance liquid chromatography, conventional chromatography, mass spectroscopy, gel electrophoresis and the like. Thus, when sufficient digestion of the unfractionated or partially fractionated heparin or heparan sulfate has taken place as determined by any one of the above techniques the heparanase is inactivated, so as to control the preponderant molecular mass of the resulting digestion products.

According to another preferred embodiment of the present invention the heparin or heparan sulfate of the lower preponderant molecular mass generated following sufficient digestion with heparanase is precipitated, e.g., by the addition of ethanol and salt and appropriate centrifugation.

According to another preferred embodiment of the present invention the heparin or heparan sulfate of the lower preponderant molecular mass generated following sufficient digestion with heparanase is size fractionated and low molecular weight heparin or heparan sulfate of a specific molecular mass range is collected. Size fractionation can be effected by any one of a variety of techniques known in the art, including, but not limited to, high performance liquid chromatography, conventional chromatography, mass spectroscopy, gel electrophoresis, differential filtration, differential centrifugation, differential dialysis and the like.

According to a preferred embodiment of the present invention the unfractionated or partially fractionated heparin or heparan sulfate used as a source material for the method of the present invention originates from any natural source such as, for heparin: porcine intestinal mucosa, bovine lung or any other tissues or organs of various animals; for heparan sulfate: bovine intestinal mucosa, porcine intestinal mucosa and bovine kidney.

In fact, the method of the present invention may be performed on an unfractionated or partially fractionated pharmaceutical grade heparin dissolved in water, or alternatively during the industrial manufacture before isolation of the pure pharmaceutical grade product. Thus, as starting material for the method of the present invention, any unfractionated or partially fractionated heparin or heparan sulfate may be used. Partially fractionated heparin or heparan sulfate can be obtained by any fractionation method known in the art, including chemical, physical and enzymatic fractionation. In order to obtain maximum efficacy of the method, it is advantageous to use heparin or heparan sulfate solutions free from suspended microparticles. The presence of these microparticles may be due to a poor dissolution of the heparin or heparan sulfate or to various impurities, or alternatively to residues of reagents used in the purification. Various solutions of heparin or heparan sulfate are hence preferably subjected to a prior filtration.

According to a preferred embodiment of the present invention digesting the unfractionated or partially fractionated heparin or heparan sulfate is continued until the lower preponderant molecular mass reaches between 1,000 and 10,000 Daltons, preferably between 3,000 and 6,000 Daltons, more preferably between 4,000 and 5,000 Daltons.

According to a preferred embodiment, the lower preponderant molecular mass reaches a state wherein at least 60% of all the constituents have a molecular mass of less than 8,000 Daltons.

Advantageous low-molecular-weight heparin or heparan sulfate according to the present invention have variable molecular mass distributions, the molecular mass of 90% of their constituents ranging between 1,000 Daltons and 10,000 Daltons, preferably between 2,000 Daltons and 9,000 Daltons more preferably between 2,000 Daltons and 8,000 Daltons, advantageously between 3,000 Daltons and 6,000 Daltons.

Another preferred low-molecular-weight heparin according to the invention has a preponderant molecular mass ranging between 1,700 Daltons and 3,300 Daltons, the molecular mass of 90% of all the constituents ranging between 1,000 Daltons and 8,000 Daltons.

The above average molecular mass is determined according to “Heparines de faible masse moleculaire” [Heparins of small molecular mass], Pharmeuropa, October 1991, 3, N. 3, pp. 161-165, and/or the proposed monograph for the European Pharmacopoeia “Heparina massae molecularis minoris”, February 1993 (PA/PH/Exp. 3T (92) 93).

The low molecular weight heparin or heparan sulfate according to the present invention preferably has anti-factor Xa activity not less than 60 IU/mg, and anti-factor Xa/anti-factor Ia activity ratio preferably not less than 1.5. The anti-factor Xa activity and the anti-factor Ia activity can be determined by methods known in the art, as for example described in U.S. Pat. No. 5,599,901, which is incorporated herein by reference. Thus, the anti-factor Xa activity and the anti-factor Xa/anti-factor IIa activity ratio are evaluated by reference to the international standard of low-molecular-weight heparins; reference WHO 1-85/600.

U.S. Pat. No. 5,714,477, which is incorporated herein by reference, teaches new compositions containing therapeutically effective amounts of heparin fragments or their derivatives, which have a bioavailability high enough to result in clinically relevant plasma levels, when administered orally or through mucous membranes or through the skin, comparable to those obtained from a subcutaneous or intramuscular injection. Such compositions are conceivable by using heparin in combination with one or several glycerol esters of fatty acids in a pharmaceutical composition.

Thus according to preferred embodiments of the present invention the low molecular weight heparin or heparan sulfate resulting from the process of the present invention is combined with one or several glycerol esters of fatty acids in a pharmaceutical composition to present bioavailability high enough to result in clinically relevant plasma levels, when administered orally or through mucous membranes or through the skin, comparable to those obtained from a subcutaneous or intramuscular injection.

U.S. Pat. No. 5,700,286, which is incorporated herein by reference, teaches a drug loaded stent which includes an expandable stent structural member, and a planar sheet of polymeric material attached to the outside of the expandable stent structural member. The polymeric material is preferably bioabsorbable, and loaded or coated with a therapeutic agent or drug, such as low molecular weight heparin, to reduce or prevent restenosis in the vessel being treated. The polymer material can be attached to the metal stent at one or more points, and wrapped in a coil around the stent in an unexpended state, to uncoil and expand in diameter to substantially match the expanded diameter of the metal stent; or can be wrapped tightly around the stent structural member and attached to itself, to stretch radially when the stent structural member is expanded.

Thus, a stent constructed as described in U.S. Pat. No. 5,700,286 and which coated with low molecular weight heparin or heparan sulfate produced by the method of the present invention is envisaged.

U.S. Pat. No. 5,686,431, which is incorporated herein by reference, teaches methods and compositions for the prevention and/or treatment of pathological processes involving the induction of TNFα secretion, e.g., allergic reaction. The compositions comprising a pharmaceutically acceptable carrier and a low molecular weight heparin. The low molecular weight heparin is present in a low effective dose and is administered at intervals of about 5-8 days wherein (i) the minimum effective dose is one-twelfth the lowest amount of the LMWH per kg of body weight that when administered to mice at intervals of about 5-8 days causes at least 50% inhibition of in vitro TNFα secretion by resting T cells and/or macrophages from said mice in response to T cell-specific antigens, mitogens, macrophage activators, disrupted extracellular matrix (dECM), laminin, or fibronectin; and (ii) the maximum effective dose is one-twelfth the greatest amount of said LMWH per kg of body weight that when administered to mice at intervals of about 5-8 days causes at least 50% inhibition of in vitro TNFα secretion by resting T cells and/or macrophages from said mice in response to T cell-specific antigens, mitogens, macrophage activators, disrupted extracellular matrix (dECM), laminin, or fibronectin.

Thus, the low molecular weight heparin produced according to the method of the present invention can be used to treat symptoms associated with over production and secretion of TNFα, such as allergies, by administering said low molecular weight heparin in dosage and repetitious rate as described in U.S. Pat. No. 5,686,431.

U.S. Pat. No. 5,686,102, which is incorporated herein by reference, teaches pharmaceutical compositions which are incorporated into the skin with the aid of microaggregates as carriers with the object to make available pharmacological active compounds in a biologically and chemically inert carrier for therapeutic and diagnostic administration to the skin or for systemic administration, and in this way to make deeper penetration into the skin or transdermal transport possible. This is effected by means of a pharmaceutical composition for topical administration, which contains asymmetric lamellar aggregates, consisting of phospholipids, pharmacological active compounds, such as low molecular weight heparin, and fluorocarbons or fluorocarbon mixtures, the proportion of fluorocarbon being in the range from 1 to 100% weight/volume, in a pharmaceutical excipient which is suitable for topical administration. Preparation is effected by emulsification of the appropriate constituents and use in ointments, creams, lotions, pastes, gels, powders or on a dressing or plaster or by means of a spray.

Thus, the low molecular weight heparin produced according to the method of the present invention can be incorporated as the pharmacological active compound in the pharmaceutical composition described in U.S. Pat. No. 5,686,102.

U.S. Pat. No. 5,639,469, which is incorporated herein by reference, teaches a device for delivering a heparinic anticoagulant across a mucosal surface in order to achieve or maintain a therapeutically effective blood level of the heparinic anticoagulant. The device involves a matrix containing a therapeutically effective amount of a heparinic anticoagulant, and a mucoadhesive or other mechanism for maintaining the matrix in contact with the mucosal surface for a time sufficient to allow release of the heparinic anticoagulant to the mucosal surface.

Thus, the low molecular weight heparin produced according to the method of the present invention can be incorporated as the heparinic anticoagulant in the device described in U.S. Pat. No. 5,686,102.

U.S. Pat. No. 5,576,304, which is incorporated herein by reference, teaches a pharmaceutical composition comprising dermatan sulfate, together with a low molecular weight heparin, free or fixed combination, which is usefull as a antithrombolytic agent with a low risk of bleeding complications.

Thus, according to a preferred embodiment a pharmaceutical composition of the present invention, includes, in addition to low molecular weight heparin produced as described herein, dermatan sulfate in a free or fixed combination, which composition is useful as a antithrombolytic agent with a low risk of bleeding complications. The composition can thus be used for increasing anti-factor Xa activity within the bloodstream of a patient in need thereof, exhibiting a synergistic enhancement of the anti-factor Xa activity with only an additive enhancement of activated partial thromboplastin time activity, such that the anti-factor Xa/activated partial thromboplastin time activity ratio of the mixture is increased as compared with either component tested alone.

U.S. Pat. No. 5,236,910, which is incorporated herein by reference, teaches a method of treatment of diabetic nephropathy which consists of administering to a living subject affected by diabetic nephropathy and exhibiting prior to treatment, thickening of the basal membrane, decrease of the glomerular anionic charges when compared to non-diabetic subjects, an effective amount of low molecular weight heparin derivatives obtained by chemical or enzymatic depolymerization, chemically modified heparin derivatives and low molecular weight dermatan sulfates obtained by chemical or enzymatic depolymerization.

Thus, according to a preferred embodiment of the present invention the low molecular weight heparin produced by the process of the present invention is employed alone or preferably in combination with dermatan sulfate for treating diabetic nephropathy and/or symptoms associated therewith, essentially as taught by U.S. Pat. No. 5,236,910.

U.S. Pat. No. 5,039,529, which is incorporated herein by reference, teaches a novel complex of (a) a metal ion such as copper, calcium, manganese, iron, and zinc ions, and (b) a fraction of heparin, heparan sulfate, low molecular weight heparin, low molecular weight heparan sulfate, heparin fragments, heparan sulfate fragments, and oligosaccharides derived from heparin or from heparan sulfate, or a salt of such fractions which fractions bind to the metal ion, wherein the complex containing from 5 to 1,000 mnole metal of component (a) per μmole of component (b). It has been found according to U.S. Pat. No. 5,039,529 that such complex exhibit markedly enhanced anti-angiogenic properties when used in conjunction with an angiostatic component, especially a steroid.

Thus, according to a preferred embodiment of the present invention the low molecular weight heparin produced by the process of the present invention is employed to form complexes thereof with either copper, calcium, manganese, iron, or zinc ions, thereby to exhibit markedly enhanced anti-angiogenic properties when used in conjunction with an angiostatic component, especially a steroid.

U.S. Pat. No. 4,731,080, which is incorporated herein by reference, teaches an improved intraocular lens which is coated with a non-smudging biologically compatible hydrophobic crosslinked vinyl-containing silicone polymer coating material, such as polymethylvinyl siloxane or polymethylphenylvinyl siloxane. The coating material is inert, does not smudge upon contact with another surface, reduces damage on contact with the intraocular tissue, particularly the endothelium, and prevents intraocular lens induction of inflammation. The coating material or matrix preferably contains at least one optically compatible medicament, such as low molecular weight heparin, which can be gradually and controllably released therefrom with time and which makes the lens suitable for implantation in both the phakic and aphakic eye. The coating material may further contain a small amount of fine particle size fumed silica.

Thus, according to a preferred embodiment of the present invention the low molecular weight heparin produced by the process of the present invention is employed as an optically compatible medicament in an intraocular lens essentially as described in U.S. Pat. No. 4,731,080.

Reference 19, which is incorporated herein by reference, teaches that low molecular weigh heparin inhibits tumor metastases, cell invasion and autoimmunity.

Thus, according to a preferred embodiment of the present invention the low molecular weight heparin produced by the process of the present invention is employed to inhibit tumor metastases, cell invasion and autoimmunity.

The cloning of the human and mouse heparanase-encoding gene, its genetic manipulation, methods of isolating and genetically manipulating homologous genes from other species, its expression in various expression systems, its purification and its natural or artificial activation (see, U.S. patent application Ser. Nos. 08/922,170; 09/071,618; 09/109,386; 09/258,892 and 09/260,038 and in PCT/US98/17954 and PCT/US98/09256) offer, for the first time, a most appropriate and reliable source of active recombinant heparanase. This enzyme may be used for producing an oligosaccharide population from unfractionated heparin or heparan sulfate. In addition to the above uses, such a population of oligosaccharides may include unique species of heparin or heparan sulfate oligosaccharides of biological or therapeutic relevance. The possibility of using an enzyme from a human source may allow production of more natural products with greater relevance for treatment of human diseases, as compared to enzymes of bacterial source such as heparinases and to physical and chemical processes. The complex nature of the heparin and heparan sulfate, and the fact that bacterial heparinase cleaves linkages only on the non-reducing side of uronic acids, while heparanase, can be specific for either of the two bonds in the repeating disaccharides, may allow the production of an oligosaccharide population containing an enormous amount of different products among which specific oligosaccharides having a relevant biological or therapeutic activity and improved pharmacological activity, may be found.

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

Reference is now made to the following examples, which together with the above descriptions, illustrate the invention in a non limiting fashion.

### EXPERIMENTAL PROCEDURES

Measurement of Unsaturated Bond Formation Using Absorbance at 232 nm:

Reactions were prepared in 1 ml quartz cuvettes. For heparinase I [Lavobacterium heparinum, Sigma, Cat. No. H2519], 3 units enzyme were added to a reaction mixture A (18 mM Tris buffer pH 7.5, 45 mM NaCl, 4 mM CaCl₂, 0.01% BSA) containing 0.2% heparin. For recombinant heparanase (expressed in insect cells, see, U.S. patent application Ser. Nos. 08/922,170; 09/071,618; 09/109,386; 09/258,892 and 09/260,038 and in PCT/US98/17954 and PCT/US98/09256, all of which are incorporated herein by reference), 5 mg enzyme were added to a reaction mixture B (20 mM phosphate citrate buffer pH 5.4, 1 mM CaCl₂, 1 mM NaCl and 0.01% BSA) containing 0.2% heparin. Absorbance at 232 nm was measured in a Cary 100 (Varian) spectrophotometer every 10 minutes for a period of 1 hour.

Heparanase and Heparinase Activity Using the Dimethylmethylene Blue (DMB) Calorimetric Assay:

100 μl heparin sepharose (50% suspension in 1×buffer A−20 mM phosphate citrate buffer pH 5.4, 1 mM CaCl₂, 1 mM NaCl) was incubated in 0.5 ml eppendorf tubes for 1 hour with enzyme preparations, in reaction mixture A for heparinase I or B for recombinant heparanase expressed in insect cells. At the end of the incubation time, the samples were centrifuged for 2 minutes at 1000 rpm. The products released to the supernatant due to the heparinase or heparanase activity were analyzed using the dimethylmethylene blue calorimetric assay (see, U.S. patent application Ser. No. 09/113,168, which is incorporated herein by reference).

To this end, supernatants (100 μl) were transferred to plastic cuvettes. The samples were diluted to 0.5 ml with PBS plus 1% BSA. 1,9-dimethylmethylene blue (32 mg dissolved in 5 ml ethanol and diluted to 1 liter with formate buffer) (0.5 ml) was added to each sample. Absorbance of the samples was determined using a spectrophotometer (Cary 100, Varian) at 530 nm. To each sample a control to which the enzyme was added at the end of the incubation time, was included.

Polyacrylamide Gel Analysis of Heparanase Products:

10 μg heparin or heparan Sulfate (Sigma) dissolved in H₂O were incubated for 17 hours at 37° C. with recombinant purified heparanase (5 μg) in a 100 μl buffer A. At the end of the incubation period, 0.25 volume of 5×glycerol loading buffer (80% glycerol, 5 mM CDTA) was added, and the samples (50 μl) were fractionated on a gradient 4-20% mini-gel in TAC buffer (40 mM Tris, 20 mM sodium acetate, 1 mM CDTA, pH-7.8). The gel was run for 45 minutes at 100 V and stained with 1,9-dimethylmethylene blue (32 mg dissolved in 5 ml ethanol and diluted to 1 liter with formate buffer). Destaining was performed with H₂O. Size of the heparanase activity products was compared to low molecular weight heparin standards with an average size of 3000 and 6000 Daltons.

### EXPERIMENTAL RESULTS

Recombinant Human Heparanase is not a Lyase:

Partially purified recombinant human heparanase expressed in insect cells was examined to determine whether it is a lyase or hydrolase. A bacterial heparinase I which is a lyase was used as a control. The formation of an unsaturated bond by lyase activity as measured by 232 nm absorbance was performed as described under experimental procedures above.

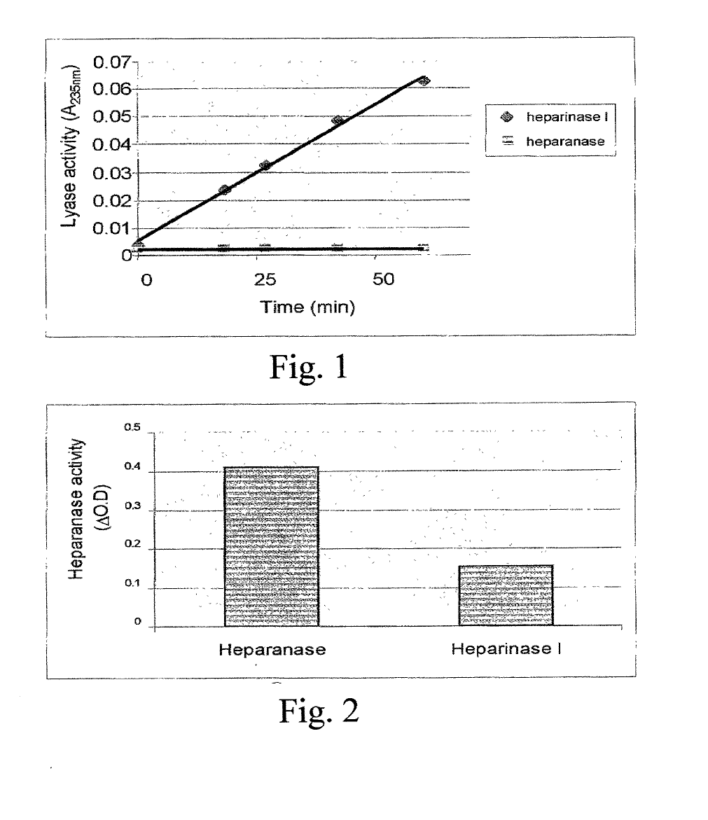

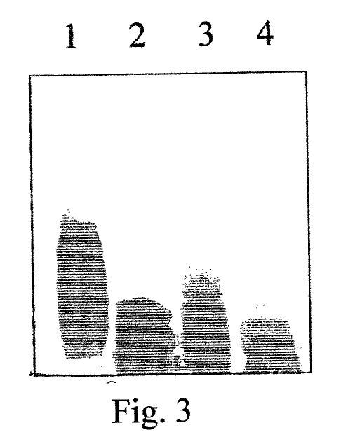

As can be seen in FIG. 1, no increase in the 232 nm absorbance was detected for heparanase, while for heparinase I a linear increase in the absorbance was found, indicating that the recombinant human heparanase is not a lyase but rather a hydrolase (see also 19). To ensure that heparanase was active during the period of time that the lyase activity was determined, the activity of the heparanase and heparinase I was determined during the same period of time using the DMB assay. As seen in FIG. 2, in the DMB assay after one hour incubation heparanase was active and its activity was even higher as compared to heparinase I activity that was incubated for the same period of time and under the same conditions.

The Products of Heparanase Activity are Smaller than 6000 Daltons:

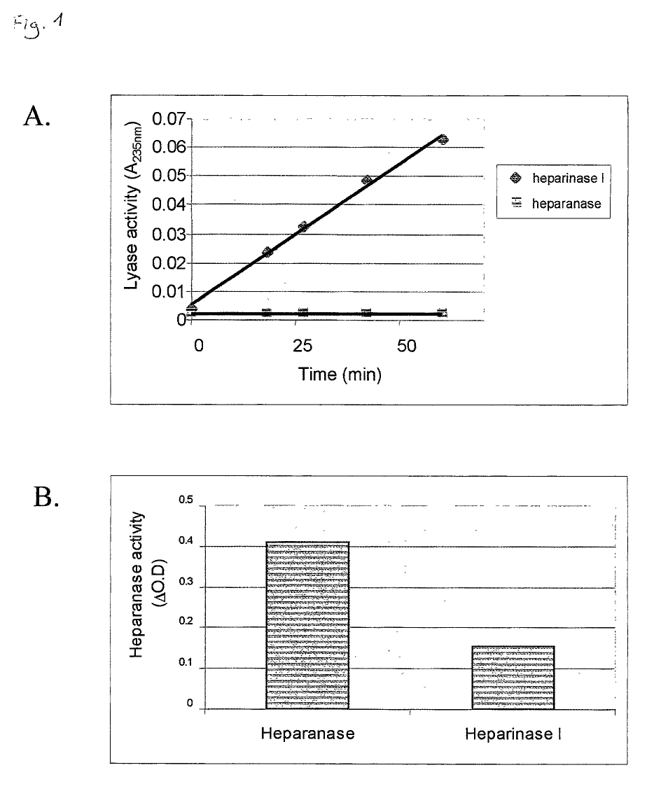

To determine the size of the heparanase products from unfractionated heparin, the enzyme was incubated with unfractionated heparin for a period of 17 hours at 37° C. The products of the heparanase reaction were fractionated on an acrylamide gel 4-20%. The size of heparanase products was compared to low molecular weight standards of heparin, 6,000 and 3,000 Daltons in average. As can be seen in FIG. 3 the size of most of the heparanase products, produced from unfractionated heparin is between 3,000 and 6,000 Daltons, however, lower product are also evident.

These results indicate that human recombinant heparanase may be used for the production of low molecular weight oligosaccharides from unfractionated heparin or heparan sulfate. As the heparanase, in contrast to the bacterial heparinase, is a hydrolase and not a lyase, the products of the enzymatic reaction catalyzed by heparanase are different from those produced by a reaction catalyzed by heparinase. As the source of the heparanase is human, the products of this enzyme may be of greater relevance for the treatment of human diseases.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications cited herein are incorporated by reference in their entirety.

### REFERENCES

1. Weitz J. I. (1997) low molecular weight heparins. New England J. Med. 4:688-698.

2. Wight, T. N., Kinsella, M. G., and Qwarnstromn, E. E. (1992). The role of proteoglycans in cell adhesion, migration and proliferation. Curr. Opin. Cell Biol., 4, 793-801.

3. Jackson, R. L., Busch, S. J., and Cardin, A. L. (1991). Glycosaminoglycans: Molecular properties, protein interactions and role in physiological processes. Physiol. Rev., 71, 481-539.

4. Wight, T. N. (1989). Cell biology of arterial proteoglycans. Arteriosclerosis, 9, 1-20.

5. Kjellen, L., and Lindahl, U. (1991). Proteoglycans: structures and interactions. Annu. Rev. Biochem., 60, 443-475.

6. Ruoslahti, E., and Yamaguchi, Y. (1991). Proteoglycans as modulators of growth factor activities. Cell, 64, 867-869.

7. Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fla.

8. Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992). Expression of heparanase by platelets and circulating cells of the immune system: Possible involvement in diapedesis and extravasation. Invasion & Metastasis, 12, 112-127.

9. Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin. Invasion & Metastasis, 14: 290-302.

10. Nakajima, M., Irimura, T., and Nicolson, G. L. (1988). Heparanase and tumor metastasis. J. Cell. Biochem., 36, 157-167.

11. Thunberg L, Backstrom G, Grundberg H, Risenfield J, and Lindahl U: The molecular size of the antithrombin-binding sequence in heparin. FEBS Lett 1980; 117:203-206.

12. Parish, C. R., Coombe, D. R., Jakobsen, K. B., and Underwood, Pa. (1987). Evidence that sulfated polysaccharides inhibit tumor metastasis by blocking tumor cell-derived heparanase. Int. J. Cancer, 40, 511-517.

13. Lider, O., Baharav, E., Mekori, Y., Miller, T.,Naparstek, Y., Vlodavsky, I. and Cohen, I. R. Suppression of experimental autoimmune diseases and prolongation of allograft survival by treatment of animals with heparinoid inhibitors of T lymphocyte heparanase. J. Clin. Invest. 83:752-756, 1989.

14. Naparstek Y. 1984 Nature 310:241-243.

15. Ernst S, Langer R, Cooney CL. and Sassisekharan R. 1995. Enzymatic degradation of glycosaminoglycans. Crit. Rev. Biochem. Mol. Biol. 30:387-444.

16. Linhardt, R. J., Galliher P. M., and Cooney C. L. 1986. Polysaccharide lyases. Appl. Biochem. Biothech. 12:135-175.

17. Linker A. and Hovin P. 1972. Heparinase and heparitinase from Flavobacteria. Meth. Enzymol. 28:902-911.

18. Goshen R., Hochberg A. A., Komer G., Levy E., Ishai-Michaeli R., Elkin M., de Groot N. and Vlodavsky I. 1996. Purification and characterization of placental heparanase and its expression by cultured cytotrophoblasts. Molecular Human Reproduction 2:679-684.

19. Pikas D. S., Li J. P., Vlodavsky I., and Lindahl U. 1998. Substrate specificity of heparanase from human hepatoma and platelets. J. Biol. Chem. 24:18770-18777.

What is claimed is:

1. A method of preparing low molecular weight heparin or low molecular weight heparan sulfate, the method comprising the step of digesting unfractionated or partially fractionated heparin or heparan sulfate having a given preponderant molecular mass with heparanase, thereby obtaining heparin or heparan sulfate of a lower preponderant molecular mass.

2. The method of claim 1, wherein said heparanase is recombinant.

3. The method of claim 1, wherein said heparanase is of human origin.

4. The method of claim 1, wherein digesting unfractionated or partially fractionated heparin or heparan sulfate is continued until said lower preponderant molecular mass reaches between 1,000 Daltons and 10,000 Daltons.

5. The method of claim 1, wherein digesting unfractionated or partially fractionated heparin or heparan sulfate is continued until said lower preponderant molecular mass reaches between 3,000 Daltons and 6,000 Daltons.

6. The method of claim 1, wherein when sufficient digestion of said unfractionated or partially fractionated heparin or heparan sulfate has taken place, said heparanase is inactivated.

7. The method of claim 1, further comprising the step of precipitating said heparin or heparan sulfate of said lower preponderant molecular mass.

8. The method of claim 7, wherein precipitation is effected by ethanol and salt.

9. The method of claim 1, further comprising the step of size fractionating said heparin or heparan sulfate of said lower preponderant molecular mass and collecting low molecular weight heparin or heparan sulfate of a specific molecular mass range.

10. The method of claim 9, wherein said specific range is selected from the group consisting of 1,000-10,000 and 3,000-6,000 Daltons for at least 90% of the heparin or heparan sulfate molecules.
